(19) United States
(12) Patent Application Publication
(10) Pub. No.: US 2026/0028298 A1
(43) Pub. Date: Jan. 29, 2026

(54) SYNTHESIS METHOD FOR HEXAFLUOROBUTADIENE AND SYNTHESIS SYSTEM FOR INTERMEDIATE DIMER

(71) Applicant: GUANGDONG HUATE GAS CO., LTD., Foshan (CN)

(72) Inventors: Junhao YANG, Foshan (CN); Yanshan CHEN, Foshan (CN); Zhuhong FU, Foshan (CN); Gaoyi RU, Foshan (CN); Wuzhong XIE, Foshan (CN); Zhiwei CHEN, Foshan (CN); Zhengzheng ZHANG, Foshan (CN)

(73) Assignee: GUANGDONG HUATE GAS CO., LTD., Foshan (CN)

(21) Appl. No.: 19/021,271

(22) Filed: Jan. 15, 2025

(30) Foreign Application Priority Data
Jul. 25, 2024 (CN) 2024110069647

(51) Int. Cl. C07C 17/281; C07C 17/42

(52) U.S. Cl. CPC C07C 17/281; C07C 17/42

(57) ABSTRACT

A synthesis method for hexafluorobutadiene is provided. In the synthesis method, a fluoride having saturated chemical bonds and having a purity ≥99.5% and a boiling point ≥120° C. as a stabilizer is uniformly mixed with chlorotrifluoroethylene and then heated for a dimerization reaction. Under the effect of the above stabilizer, the heat during the reaction can be homogenized without causing local temperature to increase easily, the flow rate of raw materials will be more stable, the occurrence of side reactions due to temperature fluctuations will be reduced, and the conversion rate of chlorotrifluoroethylene and the yield of intermediate dimer will be improved. In the method, the stabilizer and the unreacted raw materials can be separated and recycled, such that, comprehensively, the conversion rate of the chlorotrifluoroethylene can be up to 95% or higher, and the yield of the intermediate dimer can be 95% or higher.

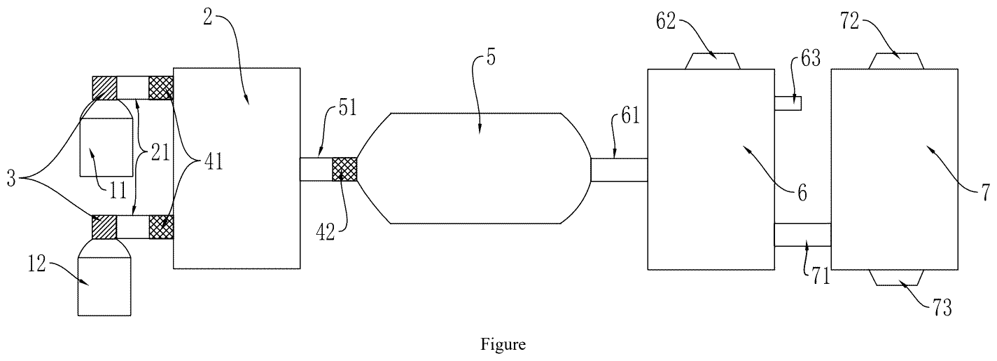

### CROSS REFERENCE TO THE RELATED APPLICATIONS

This application is based upon and claims priority to Chinese Patent Application No. 202411006964.7, filed on Jul. 25, 2024, the entire contents of which are incorporated herein by reference.

### TECHNICAL FIELD

The present invention relates to the technical field of specialty gas synthesis and, in particular, to a synthesis method for hexafluorobutadiene and a synthesis system for an intermediate dimer.

### BACKGROUND

During semiconductor chip manufacture, electronic specialty gases are widely used in silicon wafer manufacture, oxidation, lithography, vapor deposition, etching, ion implantation and other processes, and are a core product element that determines the quality of semiconductor chip products. With the rapid changes in the technology of downstream industry in recent years, a larger wafer size and a finer process technology are required. As the fineness and stability of electronic specialty gases continue to increase, the demand for medium- to high-end gas materials used in advanced processes is continuously prominent.

Electronic hexafluorobutadiene is a strategically important gas in an advanced process for 8- to 12-inch semiconductors, is used for plasma etching of semiconductors, and has a faster etching rate, a high selectivity, a high depth-to-width ratio, a short life in the atmosphere, and a negligible global warming potential compared to conventional etchants, thus being the only product of etching gases that has both application performance and environmentally friendly performance. Currently, a mainstream synthesis method for hexafluorobutadiene is as follows:

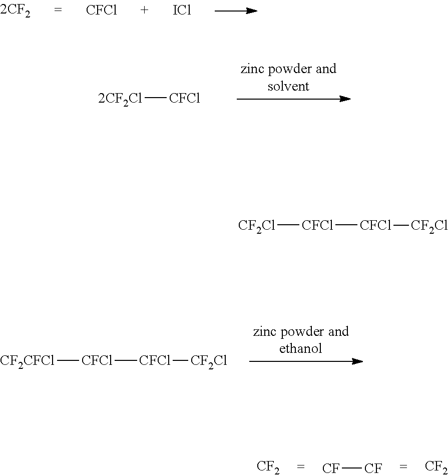

However, the aforementioned method involves more raw materials and solvents, some of which are toxic and harmful raw materials, a complex reaction process, a high cost, the separation of the intermediate dimer with greater difficulty, and a lower yield of the target product.

In order to solve the aforementioned problems, the applicant discloses, in the patent application publication no. CN 116283481 A, a synthesis method for hexafluorobutadiene, where a catalyst is improved to use in the dimerization reaction of chlorotrifluoroethylene (CTFE) to obtain an intermediate dimer and by-products, and the final CTFE conversion rate achieved in the method can reach 80%. This method has a disadvantage that the catalyst needs to be cleaned and replaced regularly, and if reaction time is long, by-products will cover the surface of the catalyst, rendering it ineffective easily. For continuous production and industrialized production operation, it is of a certain level of difficulty to clean and replace the catalyst regularly.

### SUMMARY

The main object of the present invention is to provide a synthesis method for hexafluorobutadiene so as to propose a synthesis method for hexafluorobutadiene that differs from that of background art and can achieve a comparable chlorotrifluoroethylene (CTFE) conversion rate and intermediate dimer yield without adding a catalyst in the reaction. Furthermore, the present invention provides a synthesis system for the intermediate dimer of hexafluorobutadiene, which can be used in the aforementioned synthesis method for hexafluorobutadiene to prepare the intermediate dimer.

In order to achieve the aforementioned object, the present invention provides a synthesis method for hexafluorobutadiene, which differs from the aforementioned method and includes the steps of: vaporizing a stabilizer and chlorotrifluoroethylene and then uniformly mixing the same to obtain a mixed material, heating the mixed material for dimerization reaction to obtain reaction products, separating and purifying the reaction products to obtain an intermediate dimer, and subjecting the intermediate dimer to a dechlorination post-treatment to obtain the hexafluorobutadiene, where the stabilizer is a fluoride having saturated chemical bonds and having a purity ≥99.5% and a boiling point ≥120° C.

In this solution, chlorotrifluoroethylene is used as a raw material, and a fluoride having saturated chemical bonds and having a purity ≥99.5% and a boiling point ≥120° C. is used as a stabilizer. The chlorotrifluoroethylene and the fluoride are vaporized, uniformly mixed, and heated for a dimerization reaction to obtain an intermediate dimer. After the aforementioned stabilizer is added, the heat during the reaction can be homogenized without causing the local temperature to increase easily. Meanwhile, under the effect of the stabilizer, the flow rate of raw materials will be more stable, and the occurrence of side reactions due to temperature fluctuations will be reduced. In this solution, the pressure of the reaction is generally an atmospheric pressure that does not exceed 0.2 MPa. In the present invention, in addition to a CTFE conversion rate and an intermediate dimer yield comparable to those of a method using a catalyst, the molar percentage of a main target dimer in the products is improved, achieving a good conversion effect. In particular, the conversion rate of chlorotrifluoroethylene can reach 95% or higher, the molar percentage of the main target dimer can be 60% or higher, and additionally the aforementioned stabilizer can be separated and recycled, which leads to a high economic benefit and a low system maintenance cost.

In this solution, in the case of a lower boiling point, the fluoride as a stabilizer will have a poorer stability. When the fluoride has a lower purity or has unsaturated bonds, chain-breaking will easily occur to produce new impurities at a high temperature, or self-polymerization between raw materials or reaction products will be easily induced to produce high polymer impurities, and contrarily the conversion effect will be inferior to that of a method without the addition of a stabilizer in the reaction. Compared to the prior art, no catalysts need to be used in this method, without the problems of catalyst cleaning and replacement.

In particular, the reaction products include an intermediate dimer and by-products, where the intermediate dimer is a stereoisomer of CF₂Cl—CF═CF—CF₂Cl (a main target dimer), and CF₂Cl—CFCl—CF═CF₂ (a subsidiary target dimer).

Preferably, the separation of the reaction products performed twice includes a primary separation by means of one-stage rectification to obtain unreacted raw materials for recovery and repeated reaction and a secondary separation by means of multi-stage rectification to obtain the intermediate dimer, the by-products, and the stabilizer, respectively, where the rectification is performed at a temperature of −10° C. to 100° C. Due to the larger boiling point difference and the immiscibility between the fluoride and the dimerization reaction products, the separation of the fluoride as a stabilizer from the dimerization reaction products is easier, and the use of the stabilizer and the unreacted raw materials for recycling and repeated reaction not only reduces losses and costs but also significantly improves the conversion rate of raw materials.

Preferably, the fluoride is from at least one of the compounds having a chemical general formula of C_{X}F_{2(X+1)} or C_{X}F_{(Y+2X+2)}N_{Y}, where X and Y are positive integers; or the fluoride is from at least one of perfluorononenyl trifluoroethyl ether or perfluoropolyether.

As examples only, the compounds of the molecular general formula C_{X}F_{(Y+2X+2)}N_{Y} may specifically be C₁₂F₁₈N (perfluorotributylamine), C₂₁F₄₈N₂ and the like; the compounds of the molecular general formula C_{X}F_{2(X+1)} may specifically be C₁₀F₂₂ (perfluorodecane) and the like, and an exhaustive enumeration will not be provided here.

Preferably, the fluoride has a boiling point of Z, and 120° C.≤Z≤300° C.

Preferably, in the dechlorination post-treatment, the intermediate dimer is dechlorinated in a solvent in the presence of zinc to obtain the hexafluorobutadiene. Further preferably, the solvent is ethanol. As the preferred solvent in this solution, ethanol has a better dispersing effect and will volatilize during a subsequent process without affecting the performance of reaction products or producing other impurities. Other solvents such as propanol or tetrahydrofuran may also be used in this solution.

Preferably, the heating condition is 500-700° C., and the pressure ≤0.2 MPa. When chlorotrifluoroethylene and the aforementioned stabilizer are in the aforementioned heating temperature range, the conversion effect will be better, and the occurrence of the dimerization reaction in this solution can be better guaranteed.

Further preferably, the stabilizer accounts for 20-30% (by weight) of the mixed material, and the mixed material is entered into a reactor at a flow rate of 30-80 NL/h for the dimerization reaction. When the percentage and the flow rate of the stabilizer are in the aforementioned range, the raw material conversion rate in a single pass is high, the number of cycles is small, and the energy consumption in procedures is low.

Further preferably, the heating temperature is 600-700° C., the stabilizer accounts for 20-30% (by weight) of the mixed material, and the mixed material is entered into a reactor at a flow rate of 66-80 NL/h for the dimerization reaction.

The present invention also provides a synthesis system for the intermediate dimer of hexafluorobutadiene, including a raw material storage tank, a stabilizer storage tank, a pre-mixer, a vaporizer, a first gas flow rate controller, a second gas flow rate controller, a reactor, a first rectifying column, and a second rectifying column. The first rectifying column is provided with a first material outlet and a side line extraction device at the top, and the second rectifying column is provided with a second material outlet at the top and a third material outlet at the bottom.

Both the raw material storage tank and the stabilizer storage tank are provided with a first passage in communication with the pre-mixer. Via the vaporizer, chlorotrifluoroethylene in the raw material storage tank and a fluoride in the stabilizer storage tank are vaporized, respectively. Then, the first gas flow rate controller is disposed in the first passage to control the weight ratio of the vaporized chlorotrifluoroethylene to the vaporized fluoride introduced to the pre-mixer. In the pre-mixer, the chlorotrifluoroethylene and the fluoride are uniformly mixed to obtain a mixed material.

The pre-mixer is provided with a second passage in communication with the reactor. The second gas flow rate controller is disposed in the second passage to control the flow rate of the mixed material introduced to the reactor. In the reactor, heating is performed for the dimerization reaction.

The reactor is provided with a third passage in communication with the first rectifying column, and the first rectifying column is provided with a fourth passage in communication with the second rectifying column. In the first rectifying column, reaction products are introduced for one-stage rectification, and after the one-stage rectification, unreacted raw materials are extracted from a side line at the top of the column, and light gas-phase components are outputted from the first material outlet, where the light gas-phase components mainly refer to substances such as nitrogen, oxygen, and carbon dioxide. In the second rectifying column, the remaining reaction products are introduced via the fourth passage for multi-stage rectification, and after the multi-stage rectification, an intermediate dimer product is outputted from the second material outlet, and a fluoride is outputted from the third material outlet.

Specifically, mass flow controller (MFC) is used as the gas flow rate controller.

Compared to the prior art, the technical solutions of the present invention have the following beneficial effects: In the solution, after having been vaporized, a fluoride having a purity ≥99.5% and a boiling point ≥120° C. as a stabilizer is uniformly mixed with chlorotrifluoroethylene, and then heated for the dimerization reaction. Under the effect of the above stabilizer, the heat during the reaction can be homogenized without causing local temperature to increase easily, the flow rate of raw materials will be more stable, the occurrence of side reaction due to temperature fluctuation will be reduced, and the conversion rate of chlorotrifluoroethylene and the yield of intermediate dimer will be improved. In the method, the stabilizer and the unreacted raw materials can be separated and recycled, such that, comprehensively, the conversion rate of the chlorotrifluoroethylene can be up to 95% or higher, the yield of the intermediate dimer can be 95% or higher, and the molar percentage of a main target dimer in the products is 60% or higher, thus having a good conversion effect, a high economic benefit, and a low system maintenance cost.

### BRIEF DESCRIPTION OF THE DRAWINGS

In order to more clearly illustrate the embodiments of the present application or the technical solution in the prior art, a brief introduction will be given below about the accompanying drawings needed for use in the description of embodiments or the prior art. Obviously, the accompanying drawings in the following description are merely some examples of the present application, and other relevant drawings may be obtained from these drawings without the creative effort of those skilled in the art.

FIGURE is a schematic representation of a synthesis system for a synthesis method for an intermediate dimer of hexafluorobutadiene in the present application.

The object achievement, functional features and advantages of the present application will be further described in conjunction with embodiments and with reference to accompanying drawings.

### DETAILED DESCRIPTION OF THE EMBODIMENTS

The technical solutions in the embodiments of the present invention will be described clearly and completely below, and obviously the described embodiments are not all the embodiments but merely a part of the embodiments of the present invention. On the basis of the embodiments in the present invention, all other embodiments obtained by those skilled in the art without any creative effort shall fall within the scope of protection of the present invention.

Furthermore, technical solutions between embodiments may be combined with each other on the condition that it can be realized by those skilled in the art. If the combined technical solutions contradict each other or cannot be realized, it shall be regarded that such combination of solutions does not exist, nor is it in the protection scope claimed by the present invention.

The present application provides a synthesis method for hexafluorobutadiene, including the steps of: vaporizing a stabilizer and chlorotrifluoroethylene and then uniformly mixing the same to obtain a mixed material; heating the mixed material for a dimerization reaction to obtain reaction products and produce an intermediate dimer which is a stereoisomer of CF₂Cl—CF═CF—CF₂Cl (a main target dimer) and CF₂Cl—CFCl—CF═CF₂ (a secondary target dimer) and by-products which include C—C₄F₆Cl₂, C₃F₄Cl₂, C₃FsCl, C₄F₆Cl₂, and C₂F₄Cl₂; separating and purifying the reaction products to obtain an intermediate dimer; and subjecting the intermediate dimer to dechlorination post-treatment to obtain the hexafluorobutadiene, where the stabilizer is a fluoride with a purity ≥99.5% and a boiling point ≥120° C.

In some preferred embodiments, the separation of the reaction products performed twice includes a primary separation by means of one-stage rectification to obtain unreacted raw materials for recovery and repeated reaction; and a secondary separation by means of multi-stage rectification to obtain the intermediate dimer, the by-products, and the stabilizer, respectively, where the rectification is performed at a temperature of −10° C. to 100° C.

In some preferred embodiments, the fluoride is from at least one of compounds having a chemical general formula of C_{X}F_{2(X+1)} or C_{X}F_{(Y+2X+2)}N_{Y}, where X and Y are positive integers; or the fluoride is from at least one of perfluorononenyl trifluoroethyl ether or perfluoropolyether.

In some preferred embodiments, the fluoride has a boiling point of Z, and 120° C.≤Z≤300° C.

In some preferred embodiments, during the dechlorination post-treatment, the intermediate dimer is dechlorinated in a solvent in the presence of zinc to obtain the hexafluorobutadiene. Further preferably, the solvent is ethanol or propanol or tetrahydrofuran.

In some preferred embodiment, the heating condition is 500-700° C., and the pressure ≤0.2 MPa.

In some preferred embodiments, the stabilizer accounts for 20-30% (by weight) of the mixed material, and the mixed material is entered into a reactor at a flow rate of 30-80 NL/h for the dimerization reaction.

In some preferred embodiments, the heating temperature is 600-700° C., the stabilizer accounts for 20-30% (by weight) of the mixed material, and the mixed material is entered into a reactor at a flow rate of 66-80 NL/h for the dimerization reaction.

The present application also provides a synthesis system for hexafluorobutadiene, as shown in the FIGURE, including a raw material storage tank 11, a stabilizer storage tank 12, a pre-mixer 2, a vaporizer 3, a first gas flow rate controller 41, a second gas flow rate controller 42, a reactor 5, a first rectifying column 6, and a second rectifying column 7. The first rectifying column 6 is provided with a first material outlet 62 and a side line extraction device 63 at the top, and the second rectifying column 7 is provided with a second material outlet 72 at the top and a third material outlet 73 at the bottom.

Both the raw material storage tank 11 and the stabilizer storage tank 12 are provided with a first passage 21 in communication with the pre-mixer 2. Via the vaporizer 3, chlorotrifluoroethylene in the raw material storage tank 11 and a fluoride in the stabilizer storage tank 12 are vaporized, respectively. The first gas flow rate controller 41 is disposed in the first passage 21 to control the weight ratio of the vaporized chlorotrifluoroethylene to the vaporized fluoride introduced to the pre-mixer 2. In the pre-mixer 2, the chlorotrifluoroethylene and the fluoride are uniformly mixed to obtain a mixed material.

The pre-mixer 2 is provided with a second passage 51 in communication with the reactor 5. The second gas flow rate controller 42 is disposed in the second passage 51 to control the flow rate of the mixed material introduced to the reactor 5. In the reactor 5, heating is performed for the dimerization reaction.

The reactor 5 is provided with a third passage 61 in communication with the first rectifying column 6, and the first rectifying column 6 is provided with a fourth passage 71 in communication with the second rectifying column 7. In the first rectifying column 6, reaction products are introduced for one-stage rectification, and after the one-stage rectification, unreacted raw materials are extracted from a side line at the top of the column, and light gas-phase components are outputted from the first material outlet 62, where the light gas-phase components mainly refer to substances such as nitrogen, oxygen, and carbon dioxide. In the second rectifying column 7, the remaining reaction products are introduced via the fourth passage 71 for multi-stage rectification, and after the multi-stage rectification, an intermediate dimer product is outputted from the second material outlet 72, and a fluoride is outputted from the third material outlet 73.

The technical solutions of the present invention will be further described in detail below in combination with specific examples, and it should be understood that the following examples are only used to illustrate the present invention and are not intended to limit the present invention.

### Example 1-1

A synthesis method for hexafluorobutadiene was provided, which includes the following steps.

CTFE and a stabilizer were vaporized and then uniformly mixed at a weight ratio controlled by an MFC in a pre-mixer so as to obtain a mixed material. The mixed material was introduced to a reactor at a certain flow rate and heated at 400° C. for a dimerization reaction, so as to produce an intermediate dimer and by-products, where the intermediate dimer is a stereoisomer of CF₂Cl—CF═CF—CF₂Cl, and CF₂Cl—CFCl—CF═CF₂.

After the completion of the reaction, multi-stage rectification was performed at a rectification temperature of −10-100° C., so as to obtain an intermediate dimer, by-products, and a stabilizer, respectively, where the intermediate dimer is a stereoisomer of CF₂Cl—CF═CF—CF₂Cl, and CF₂Cl—CFCl—CF═CF₂. The stabilizer was recovered and recycled, the intermediate dimer was mixed with an ethanol solvent, and a zinc powder was added for dechlorination, so as to obtain the hexafluorobutadiene.

The stabilizer contains a perfluorotributylamine (abbreviated to FC-43 in the following examples, with a chemical formula of C₁₂F₁₈N and a boiling point of 120° C. or higher) with a purity of 99.5%. The stabilizer accounts for 17% (by weight) of the mixed material, and the mixed material was introduced to the reactor at a flow rate of 22 NL/h.

### Example 1-2

A synthesis method for hexafluorobutadiene was provided, which includes the following steps.

CTFE and a stabilizer were vaporized and then uniformly mixed at a weight ratio controlled by an MFC in a pre-mixer so as to obtain a mixed material. The mixed material was introduced to a reactor at a certain flow rate and heated at 700° C. for a dimerization reaction, so as to produce an intermediate dimer and by-products, where the intermediate dimer is a stereoisomer of CF₂Cl—CF═CF═CF₂Cl, and CF₂Cl—CFCl—CF═CF₂.

After the completion of the reaction, multi-stage rectification was performed at a rectification temperature of −10-100° C., so as to obtain an intermediate dimer, by-products, and a stabilizer, respectively, where the intermediate dimer is a stereoisomer of CF₂Cl—CF═CF—CF₂Cl, and CF₂Cl—CFCl—CF═CF₂. The stabilizer was recovered and recycled, the intermediate dimer was mixed with an ethanol solvent, and a zinc powder was added for dechlorination, so as to obtain the hexafluorobutadiene.

The stabilizer contains a C₂₁F₄₈N₂ (abbreviated to FC-40 in the following examples, with a boiling point of 120° C. or higher) with a purity of 99.5%. The stabilizer accounts for 20% (by weight) of the mixed material, and the mixed material was introduced to the reactor at a flow rate of 80 NL/h.

### Example 1-3

A synthesis method for hexafluorobutadiene was provided, which includes the following steps.

CTFE and a stabilizer were vaporized and then uniformly mixed at a weight ratio controlled by an MFC in a pre-mixer so as to obtain a mixed material. The mixed material was introduced to a reactor at a certain flow rate and heated at 500° C. for a dimerization reaction, so as to produce an intermediate dimer and by-products, where the intermediate dimer is a stereoisomer of CF₂Cl—CF═CF═CF₂Cl, and CF₂Cl—CFCl—CF═CF₂.

After the completion of the reaction, multi-stage rectification was performed at a rectification temperature of −10-100° C., so as to obtain an intermediate dimer, by-products, and a stabilizer, respectively, where the intermediate dimer is a stereoisomer of CF₂Cl—CF═CF—CF₂Cl, and CF₂Cl—CFCl—CF═CF₂. The stabilizer was recovered and recycled, the intermediate dimer was mixed with an ethanol solvent, and a zinc powder was added for dechlorination, so as to obtain the hexafluorobutadiene.

The stabilizer contains an FC-43 with a purity of 99.8%. The stabilizer accounts for 30% (by weight) of the mixed material, and the mixed material was introduced to the reactor at a flow rate of 30 NL/h.

The conversion rate of CTFE and the yield of intermediate dimer after the reaction in the examples above were tested, and the data were shown in the following table.

| | Conversion | Yield of | Molar ratio of reaction products | | | |
|---|---|---|---|---|---|---|
| | rate of | intermediate | Main target | Secondary | | Remaining |
| Example no. | CTFE | dimer | dimer | target dimer | C—C₄F₆Cl₂ | by-products |
| Example 1-1 | 40% | 38% | 25% | 4% | 40% | 26% |
| Example 1-2 | 65% | 80% | 42% | 6% | 22% | 30% |
| Example 1-3 | 52% | 68% | 36% | 3% | 28% | 33% |

Note: The volume ratio of by-product C—C₄F₆Cl₂ was given, and the main reason is that, as one of the impurities that have been the focus of attention, C—C₄F₆Cl₂ has a similar property to that of target dimers and cannot be separated easily. Therefore, it is required in this solution that the volume ratio of this by-product after the dimerization reaction should be relatively small and that the separation of the remaining by-products should be relatively easy.

As can be seen from the test results in the table above, in example 1, even though the weight percentage of stabilizer in mixed material, the heating temperature for the dimerization reaction, and the flow rate of introduced mixed material were not limited to a preferred range, a CTFE conversion rate of 40% or higher can be achieved. However, due to the smaller flow rate and lower reaction temperature in example 1, the conversion effect was relatively average. In example 1-2 and example 1-3, both the flow rate and the reaction temperature were limited to a preferred range, and consequently both the conversion rate of CTFE and the yields of intermediate dimer and main target dimer were significantly improved.

Furthermore, in addition to the exemplary FC-40 and FC-43, which are mentioned in the examples and used as a stabilizer, other fluorides with a boiling point ≥120° C., such as at least one of the compounds with a chemical general formula conforming to C_{X}F_{2(X+1)} or C_{X}F_{(Y+2X+2)}N_{Y}, or at least one of perfluorononenyl trifluoroethyl ether or perfluoropolyether, when having a purity greater than 99.5%, can at least enable the CTFE conversion rate to reach 40% or higher. Additionally, the CTFE conversion rate can be further improved by recovering the unreacted raw materials.

### Example 2

Hexafluorobutadiene was prepared using a synthesis method similar to that in example 1-2, except that the heating temperature was different, where the stabilizer contained an FC-40 with a purity of 99.5%.

The adjustments in heating temperature in this example were as shown in the following table, and the conversion rate of CTFE, the yield of intermediate dimer, and the like after the reaction were tested, and the resulting data were as shown in the following table.

| | Heating | Conversion | Yield of | Molar ratio of reaction products | | | |
|---|---|---|---|---|---|---|---|
| | temperature/ | rate of | intermediate | Main target | Secondary | | Remaining |
| Example no. | ° C. | CTFE | dimer | dimer | target dimer | C—C₄F₆Cl₂ | by-products |
| Example 1-2 | 700 | 65% | 80% | 42% | 6% | 22% | 30% |
| Example 2-1 | 400 | 40% | 35% | 20% | 2% | 30% | 48% |
| Example 2-2 | 450 | 43% | 40% | 25% | 4% | 35% | 36% |
| Example 2-3 | 500 | 50% | 65% | 38% | 5% | 30% | 27% |
| Example 2-4 | 600 | 60% | 80% | 42% | 5% | 28% | 25% |
| Example 2-5 | 650 | 68% | 82% | 45% | 7% | 20% | 28% |
| Example 2-6 | 750 | 62% | 62% | 34% | 3% | 26% | 37% |
| Example 2-7 | 800 | 60% | 55% | 32% | 2% | 28% | 38% |

As can be seen from the test results in the table above, the conversion effect of hexafluorobutadiene can be further improved by adjusting the heating temperature during the dimerization reaction. As can be seen from the aforementioned example data, the conversion rate of CTFE and the yield of intermediate dimer product show a tendency to increase first and decrease later with the increase of heating temperature. In the aforementioned example, the optimum was reached at 650° C., the change started and showed a tendency to decline at 650° C.-700° C., and the yield of intermediate dimer decreased significantly from 80% to 62% at 700° C.-750° C. Therefore, the heating temperature that has a better effect on the synthesis of the product is 500-700° C., at which the conversion rate of CTFE is ≥50%, and the yield of intermediate dimer is 65% or higher.

### Example 3

Furthermore, this solution also provided a set of examples with better performance, where each of the parameters was in a preferred range.

A synthesis method for hexafluorobutadiene was provided, which includes the following steps.

CTFE and a stabilizer were vaporized and then uniformly mixed at a weight ratio controlled by an MFC in a pre-mixer so as to obtain a mixed material. The mixed material was introduced to a reactor at a certain flow rate and heated at 620° C. for a dimerization reaction, so as to produce an intermediate dimer and by-products, where the intermediate dimer is a stereoisomer of CF₂Cl—CF═CF═CF₂Cl, and CF₂Cl—CFCl—CF═CF₂.

After the completion of the reaction, multi-stage rectification was performed at a rectification temperature of −10-100° C., so as to obtain an intermediate dimer, by-products, and a stabilizer, respectively, where the intermediate dimer is a stereoisomer of CF₂Cl—CF═CF—CF₂Cl, and CF₂Cl—CFCl—CF═CF₂. The stabilizer was recovered and recycled, the intermediate dimer was mixed with an ethanol solvent, and a zinc powder was added for dechlorination, so as to obtain the hexafluorobutadiene.

The stabilizer contains an FC-40 with a purity of 99.5%. The stabilizer accounts for 25% (by weight) of the mixed material, and the mixed material was introduced to the reactor at a flow rate of 66 NL/h.

### Example 4

Hexafluorobutadiene was prepared using a synthesis method similar to that of example 3, except that the separation of dimerization reaction products performed twice includes a primary separation by means of one-stage rectification to obtain unreacted raw materials and return to the repeated reaction and a secondary separation by means of multi-stage rectification to obtain an intermediate dimer, by-products, and a stabilizer, respectively.

### Comparative Example 1

Hexafluorobutadiene was prepared using a synthesis method similar to that of example 5, except that the material introduced to the reactor only contained CTFE, with no stabilizer.

### Comparative Example 2

The preferred example 6 of the published patent CN 116283481 A was used as comparative example 2.

A synthesis method for hexafluorobutadiene was provided, which includes the following steps. Under catalysis and heating, the dimerization reaction of chlorotrifluoroethylene was performed in a reactor so as to produce a dimer and by-products, where the dimer is a stereoisomer of CF₂Cl—CF═CF—CF₂Cl, and CF₂Cl—CFCl—CF═CF₂. Then, rectification and purification were performed at room temperature to 100° C. so as to obtain the stereoisomer of CF₂Cl—CF═CF—CF₂Cl, and CF₂Cl—CFCl—CF═CF₂. Then, a zinc powder was added, and the dimer was dechlorinated in an ethanol solvent so as to obtain the hexafluorobutadiene. The flow rate of the chlorotrifluoroethylene introduced to the reactor was 50 NL/h, the temperature during heating was 630° C., and the amount of the catalyst filling the reactor was ⅓.

The preparation of the catalyst includes the following steps. Alumina, nickel fluoride, and metallic nickel were stirred in ethanol for 10 h and subjected to granulation and calcination so as to obtain the catalyst. The temperature during the calcination was 500° C., and the size of the catalyst after granulation was 3 mm×3 mm. The weight ratio of nickel fluoride to metallic nickel in the catalyst was 5:1.

### Comparative Example 3

Hexafluorobutadiene was prepared using a synthesis method similar to that of example 5, except that the ingredient of the stabilizer had a purity of 95%.

The conversion rate of CTFE and the yield of intermediate dimer after reaction in the examples and comparative examples above were tested, and the resulting data were as shown in the following table.

| | Conversion | Yield of | Molar ratio of reaction products | | | |
|---|---|---|---|---|---|---|
| | rate of | intermediate | Main target | Secondary | | Remaining |
| Example no. | CTFE | dimer | dimer | target dimer | C—C₄F₆Cl₂ | by-products |
| Example 3 | 80% | 95% | 58% | 6% | 15% | 21% |
| Example 4 | 95% | 95% | 60% | 5% | 15% | 20% |
| Comparative | 45% | 50% | 35% | 4% | 38% | 23% |
| example 1 | | | | | | |
| Comparative | 82% | 95% | 55% | 8% | 16% | 21% |
| example 2 | | | | | | |
| Comparative | 50% | 40% | 25% | 2% | 30% | 45% |
| example 3 | | | | | | |

As can be seen from the test results in the table above, when a further limitation was set on each of the parameters, specifically, the preferable heating temperature of 600-700° C., the flow rate of the mixed material introduced to the reactor of 66-80 NL/h, and the weight percentage of stabilizer in mixed material of 20-30%, the corresponding CTFE conversion rate and intermediate dimer yield were 60% or higher, and the percentage of the main target dimer was 80% or higher.

Example 3 is a preferred example. When the heating temperature was 620° C., the flow rate of the mixed material introduced to the reactor was 66 NL/h, and the weight percentage of stabilizer in the mixed material was 25%, the conversion rate of CTFE reached 80%, the yield of intermediate dimer reached 95%, and the molar percentage of the main target dimer was 58%, which was comparable to the effect of the catalyst in comparative example 2. Additionally, the molar percentage of the main target dimer in this solution was higher. Example 4 is the most preferred example, in which the separated unreacted raw material was added for repeated reaction, such that the conversion rate of CTFE was increased to 95%, the yield of intermediate dimer reached 95%, and the molar percentage of the main target dimer reached 60%.

As can be seen from the test results of comparative examples 1-3, when the stabilizer was not added, the corresponding CTFE conversion rate and intermediate dimer (main target dimer and secondary target dimer) yield decreased significantly. When the purity of the stabilizer was lower than 99.5%, chain-breaking would easily occur to produce new impurities at a high temperature, which is detrimental to the subsequent synthesis of hexafluorobutadiene. For example, in comparative example 3, when the purity of the stabilizer was 95%, the yield of intermediate dimer was even lower than that of comparative example 1 without the addition of the stabilizer.

As can be seen from the test results of the examples and comparative examples above, the use of the stabilizer solution of the present application not only helps to achieve a CTFE conversion rate and an intermediate dimer yield comparable to those of a solution using a catalyst but also improves the yield of the main target dimer, thus having a good conversion effect. Additionally, the separated unreacted raw materials were recycled and added to the repeated reaction, which also significantly improved the conversion rate of CTFE.

The foregoing description is merely preferred examples of the present invention, without intention to limit the scope of the present invention, and any equivalent structural transformation made under the inventive concept of the present invention using the contents of the present specification, or direct/indirect application in other relevant technical fields will fall within the protection scope of the present invention.

What is claimed is:

1. A synthesis method for hexafluorobutadiene, comprising the steps of: vaporizing a stabilizer and chlorotrifluoroethylene and then uniformly mixing the same to obtain a mixed material, heating the mixed material for a dimerization reaction to obtain reaction products, separating and purifying the reaction products to obtain an intermediate dimer, and subjecting the intermediate dimer to a dechlorination post-treatment to obtain the hexafluorobutadiene,
wherein the stabilizer is a fluoride having saturated chemical bonds and having a purity ≥99.5% and a boiling point ≥120° C.

2. The synthesis method for the hexafluorobutadiene according to claim 1, wherein the reaction products comprise the intermediate dimer and by-products, and the intermediate dimer is a stereoisomer of CF₂Cl—CF═CF—CF₂Cl, and CF₂Cl—CFCl—CF═CF₂.

3. The synthesis method for the hexafluorobutadiene according to claim 2, wherein the separation of the reaction products performed twice comprises a primary separation by means of one-stage rectification to obtain unreacted raw materials for recovery and repeated reaction; and a secondary separation by means of multi-stage rectification to obtain the intermediate dimer, the by-products, and the stabilizer, respectively, wherein the one-stage rectification and the multi-stage rectification are performed at a temperature of −10° C. to 100° C.

4. The synthesis method for the hexafluorobutadiene according to claim 1, wherein the fluoride is from at least one of compounds having a chemical general formula of C_{X}F_{2(X+1)} or C_{X}F_{(Y+2X+2)}N_{Y}, wherein X and Y are positive integers;
or the fluoride is from at least one of perfluorononenyl trifluoroethyl ether or perfluoropolyether.

5. The synthesis method for the hexafluorobutadiene according to claim 1, wherein the fluoride has a boiling point of Z, and 120° C.≤Z≤300° C.

6. The synthesis method for the hexafluorobutadiene according to claim 1, wherein in the dechlorination post-treatment, the intermediate dimer is dechlorinated in a solvent in a presence of zinc to obtain the hexafluorobutadiene.

7. The synthesis method for the hexafluorobutadiene according to claim 1, wherein a heating condition is 500-700° C. and a pressure ≤0.2 MPa.

8. The synthesis method for the hexafluorobutadiene according to claim 7, wherein a weight of the stabilizer accounts for 20-30% of a weight of the mixed material, and the mixed material is entered into a reactor at a flow rate of 30-80 NL/h for the dimerization reaction.

9. The synthesis method for the hexafluorobutadiene according to claim 7, wherein a heating temperature is 600-700° C., a weight of the stabilizer accounts for 20-30% of a weight of the mixed material, and the mixed material is entered into a reactor at a flow rate of 66-80 NL/h for the dimerization reaction.

10. A synthesis system for an intermediate dimer of hexafluorobutadiene, comprising a raw material storage tank, a stabilizer storage tank, a pre-mixer, a vaporizer, a first gas flow rate controller, a second gas flow rate controller, a reactor, a first rectifying column, and a second rectifying column, wherein the first rectifying column is provided with a first material outlet and a side line extraction device at a top, and the second rectifying column is provided with a second material outlet at a top and a third material outlet at a bottom;
both the raw material storage tank and the stabilizer storage tank are provided with a first passage in communication with the pre-mixer; via the vaporizer, chlorotrifluoroethylene in the raw material storage tank and a fluoride in the stabilizer storage tank are vaporized, respectively; the first gas flow rate controller is disposed in the first passage to control a weight ratio of vaporized chlorotrifluoroethylene to a vaporized fluoride introduced to the pre-mixer; in the pre-mixer, the vaporized chlorotrifluoroethylene and the vaporized fluoride are uniformly mixed to obtain a mixed material;
the pre-mixer is provided with a second passage in communication with the reactor; the second gas flow rate controller is disposed in the second passage to control a flow rate of the mixed material introduced to the reactor; in the reactor, heating is performed for a dimerization reaction;
the reactor is provided with a third passage in communication with the first rectifying column, and the first rectifying column is provided with a fourth passage in communication with the second rectifying column; in the first rectifying column, reaction products are introduced for one-stage rectification, and after the one-stage rectification, unreacted raw materials are extracted from a side line at the top of the first rectifying column, and light gas-phase components are outputted from the first material outlet; and in the second rectifying column, remaining reaction products are introduced via the fourth passage for multi-stage rectification, and after the multi-stage rectification, an intermediate dimer product is outputted from the second material outlet, and the fluoride is outputted from the third material outlet.
